(19) United States
(12) Patent Application Publication
(10) Pub. No.: US 2005/0283106 A1
(43) Pub. Date: Dec. 22, 2005

(54) Reusable support device with therapeutic inserts

(76) Inventors: Shane Smith, Chattanooga, TN (US); Joseph Czerwinski, Chattanooga, TN (US); Don Moak, Chattanooga, TN (US); Blair Ramey, Chattanooga, TN (US); Bob Clausen, Chattanooga, TN (US); Fred Hadtke, New York, NY (US); Jodi Yarnoff, New York, NY (US)

Correspondence Address: DOUGLAS T. JOHNSON;MILLER & MARTIN, 1000 VOLUNTEER BUILDING, 832 GEORGIA AVENUE, CHATTANOOGA, TN, 37402-2289, US

(21) Appl. No.: 11/154,342

(22) Filed: Jun. 16, 2005

Related U.S. Application Data
(60) Provisional application No. 60/580,615, filed on Jun. 17, 2004.

(51) Int. Cl. A61F 13/00; A61F 13/06

(52) U.S. Cl. 602/60; 602/61

(57) ABSTRACT

A reusable support device is provided with an open web section to permit interface of therapeutic delivery devices with the wearer and to thereby permit the dispensing of medicaments or the direct application of other therapeutic treatment. The open web section allows communication with the wearer's skin and the treatment delivery devices may be positioned by engagement with the web, and secured with an overwrap. Therapeutic delivery devices may be inserted and removed from supports without removing the support devices from the wearer.

The present application claims priority from U.S. Provisional Application No. 60/580,615 filed Jun. 17, 2004.

### FIELD OF THE INVENTION

The present invention relates generally to therapeutic support braces, and more particularly to an improved construction and design of wearable braces to provide both therapy and support.

### BACKGROUND OF THE INVENTION

Orthopedic compression bandages, braces, and sleeves have long been employed to provide support for athletic and medical purposes. These are commonly worn over the wrist, elbows, knees and ankles, and may also be employed on lower legs and forearms, and less frequently on upper legs and arms. In addition, braces are often worn to support the wearer's back. Compression devices may provide support during normal movement which may be especially required by persons recovering from injuries. Compression devices also provide support for ligaments, tendons, muscles and joints against the stresses of over-extension which may occur during exercise. In addition, wound dressings have often been used to position absorbent material adjacent to injured tissue or in some cases to apply antibiotic or moisturizing ointments and lotions. Wound dressings have lacked the ability to provide support, and most orthopedic compression devices have not possessed the ability to provide therapeutic action.

One attempt to overcome the shortcoming of therapeutic braces was provided by Williams, U.S. Pat. No. 5,415,624, which discloses a wearable therapeutic brace having pockets that can receive insertable hot and cold packs. However, this brace must be removed in order to insert the temperature management packs and does not permit the therapeutic packs to contact the wearer's skin. A variety of heat wraps and adhesive heating patches have also been offered, however, such wraps generally fail to provide one or more of therapeutic support, the ability to change the therapeutic pack without removing the product, or contact of the therapeutic pack with the wearer's skin.

### SUMMARY OF THE INVENTION

The present invention resolves the shortcomings of the prior art devices and provides a greatly improved therapeutic brace which is comfortable, permits use with a variety of therapeutic packs, including those requiring contact with the wearer's skin to administer medicaments or therapeutic relief, and in some embodiments permits changing of therapeutic packs without removing the brace from the wearer's body.

The reusable support device provides generally a stretchable sleeve in its most preferred embodiments adapted to be fitted on the wearer's leg, arm, wrist, knee, ankle or torso that has a relatively open or webbed section adapted to receive a therapeutic pack. The sleeve also has a strap that may be designed to accommodate therapeutic packs and which overlays the open section of the sleeve providing additional support to that section and allowing the wearer to adjust the compression provided by the sleeve. In a less preferred embodiment, the support device may constitute only a wrap, but with specially designed and webbed pockets to hold therapeutic inserts.

### OBJECTS AND ADVANTAGES OF THE INVENTION

A principal object and advantage of the present invention is to provide a reusable compression support device for the ankle, knee, arm, wrist, elbow, and back that is capable of receiving an insert to administer medicaments such as topical analgesic or temperature relief such as heat or cold.

It is a further object of the invention to provide a support sleeve having openings to permit a therapeutic pack placed in the sleeve to communicate directly with the wearer's skin.

It is yet another object of the invention to provide therapeutic packs designed to interface with openings in the support sleeve.

It is yet another object of the invention to provide a support sleeve permitting the insertion and removal of therapeutic packs without the necessity of removing the sleeve from the wearer's body.

Other objects and advantages of the invention will become apparent from the following description taken in conjunction with the accompanying drawings wherein are shown by way of illustration and examples certain embodiments of the invention.

### BRIEF DESCRIPTION OF THE DRAWING

FIG. 1 is a view of the support device positioned on a wearer's knee ready for acceptance of a therapeutic delivery device.

FIG. 2 is the support device of FIG. 1 in its closed position after any desired therapeutic delivery device has been installed.

FIG. 3 is a photograph of the support device configured for the wearer's knee showing a sponge element in the place of a therapeutic delivery device.

FIG. 4 shows the knee support of FIG. 3 in its closed position.

FIG. 5 shows the knee support of FIGS. 3 and 4 in use.

FIG. 6 illustrates the positioning of a cold temperature therapeutic delivery device in the overwrap portion of the knee support device of FIG. 1.

FIG. 7 illustrates the positioning of a hot temperature therapeutic delivery device in the overwrap portion of the knee support device of FIG. 1.

FIG. 8a shows a support device according to the present invention adapted for use on the wearer's back ready to receive therapeutic delivery device.

FIG. 8b shows an alternate support device according to the present invention adapted for use on the wearer's back in a wrap form with a pocket to receive therapeutic inserts.

FIG. 9 is a photograph of a support device intended for use on a wearer's back having an alternative open configuration to receive therapeutic delivery devices.

FIG. 10 illustrates a support device for an ankle according to the present invention in an open position ready to receive a therapeutic delivery device.

FIG. 11 illustrates a support device for an arm or elbow according to the present invention in an open position ready to receive a therapeutic delivery device.

FIG. 12 illustrates a support device for a wrist according to the present invention in an open position ready to receive a therapeutic delivery device.

FIG. 13 is a photograph of a support device for a wearer's wrist in an open position ready to receive a therapeutic delivery device.

FIG. 14 is a photograph of the wrist support device of FIG. 13 in its closed position.

FIG. 15a is a top view of a web structure particularly suitable for use with a knee support according to the present invention.

FIG. 15b is a side sectional view of the web structure of FIG. 15A.

FIG. 16 is a top plan view of a web structure particularly suitable for use in an elbow support device according to the present invention.

FIG. 17 is a top plan view of a web structure particularly suitable for use in an ankle support device according to the present invention.

FIG. 18 is a top plan view of a web structure particularly suitable for use in a wrist support device according to the present invention.

FIG. 19 is a perspective illustration of the web structure of FIG. 15a illustrating alternative constructions of the web and alternative systems to interface with therapeutic delivery devices.

FIG. 20 is a perspective view of a support device adapted for the knee, elbow, arm or leg prior to sealing the ends of the body sheet to form a sleeve and showing details of possible structure and manufacture of the support.

FIG. 21 illustrates a configuration for a therapeutic delivery device that can mechanically attach to the web structure located in the support.

FIG. 22 illustrates an alternative structure for the web structure and therapeutic delivery devices.

FIGS. 23a-23d depict a medicament dispensing device adapted for use in the invention.

FIG. 24 illustrates an alternative design for an ankle support of the invention.

FIG. 25 illustrates the ankle support of FIG. 24 in a closed position.

### DETAILED DESCRIPTION OF THE INVENTION

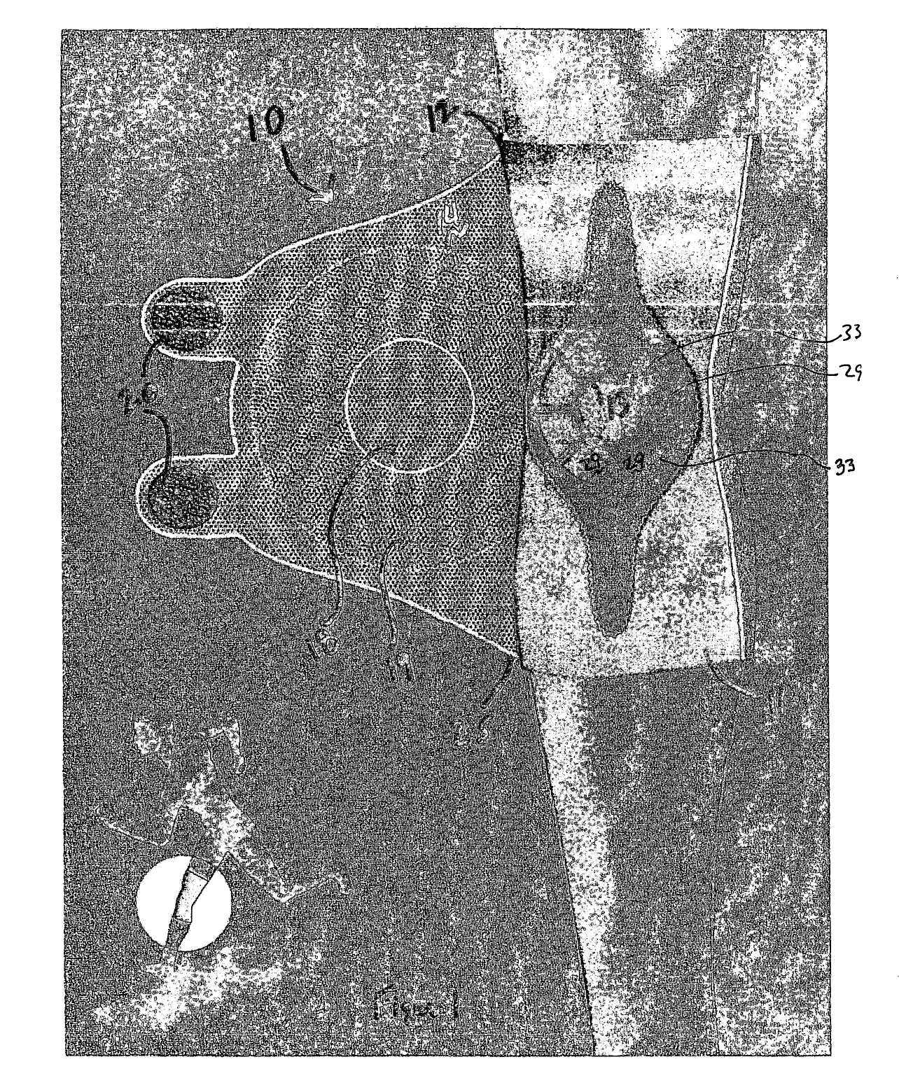

The invention may be explained with reference to FIG. 1 which depicts a knee support 10 according to the present invention having a brace body 11 rolled into a sleeve 12 that can receive a wearer's leg. Located within the brace body 11 is web 13 having ribs 33 separating apertures 29. The configuration of web 13 may take many forms but is designed to provide a structure that helps to position or secure therapeutic delivery devices such as medicament dispensing inserts and also to permit communication between such devices and the wearer's skin. In addition, the web 13 may provide structural integrity to the sleeve. Also shown is overwrap 14 which has an edge 25 secured to sleeve 12 and an opposite end with securing means such as hook fasteners 20 that will attach to the nappy surface or landing zones of brace body 11. The intermediate section of overwrap 14 may have a cupped depression 18 preferably covered by a liner 19 to form a pocket. Therapeutic delivery devices may also be inserted in cup 18 and held in position by liner 19. Alternatively, the overwrap 14 may operate without liner 19 and therapeutic delivery devices may be held in place by pressure of overwrap 14 exerted against the web 13 when fastened. Thus, the sleeve 12 forms a lumen to receive a wearer's limb or joint. The lumen wall has a web section 13. On a first side of the web section 13, an overwrap 14 is joined to the lumen wall. Preferably, the overwrap 14 is permanently joined at this first side, as by seam 25. Alternatively, overwrap 14 may be releasably joined as by hook and loop fasteners. The overwrap 14 extends from the first side across the web section 13 to the opposite second side of the web section and is there releasably secured by fastener 20 either to the lumen wall, or if completely encircling the sleeve, the fastener 20 may attach to the overwrap 14 itself.

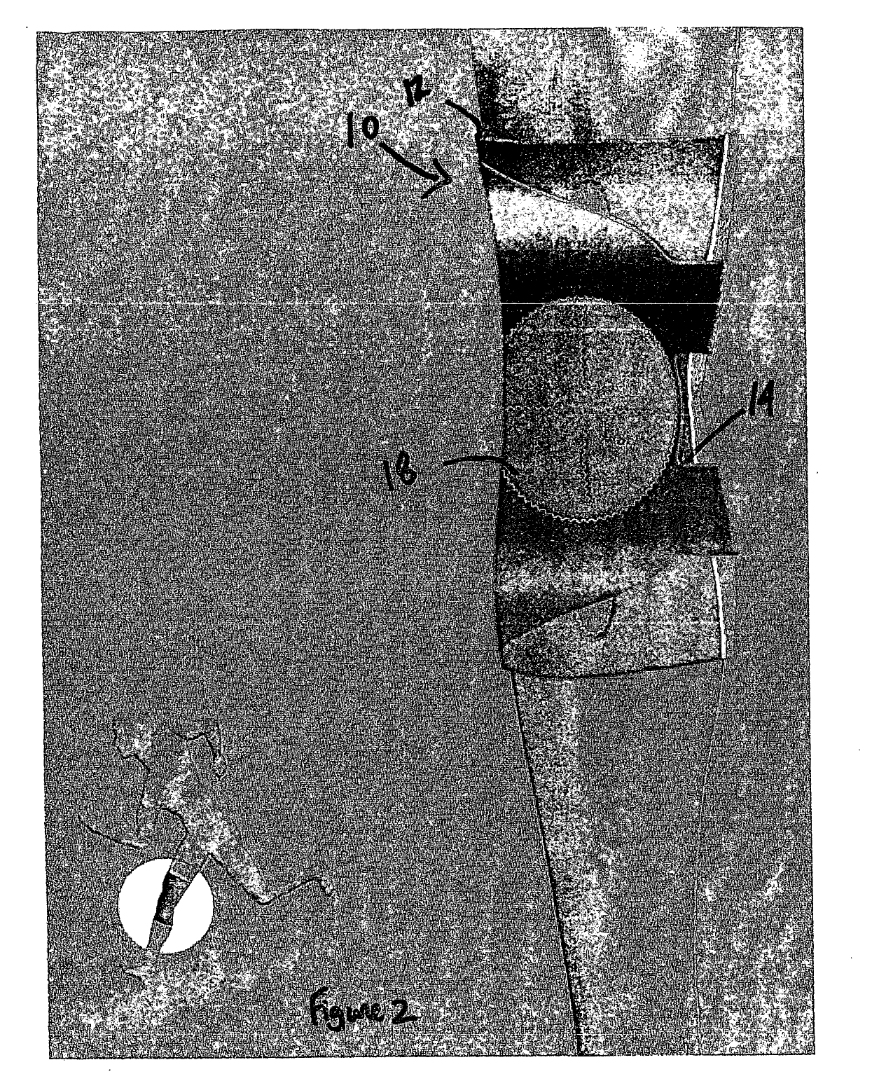

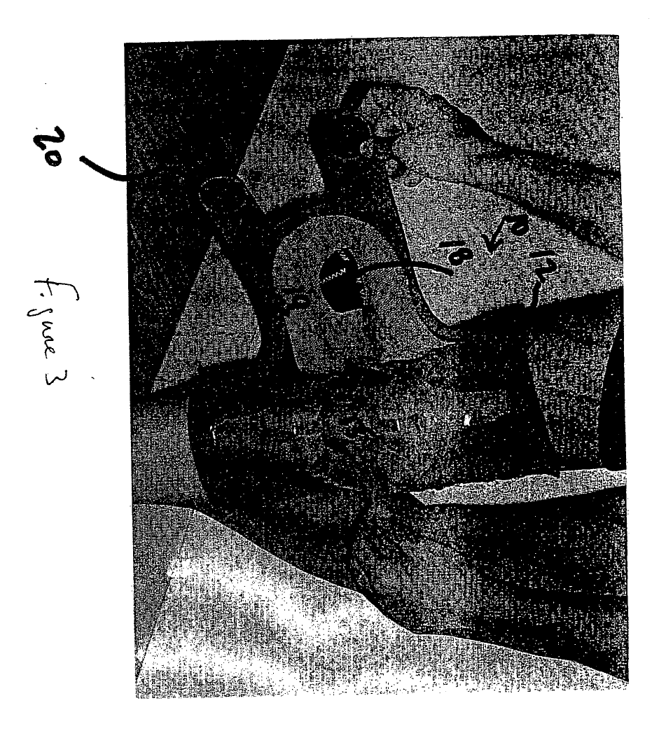

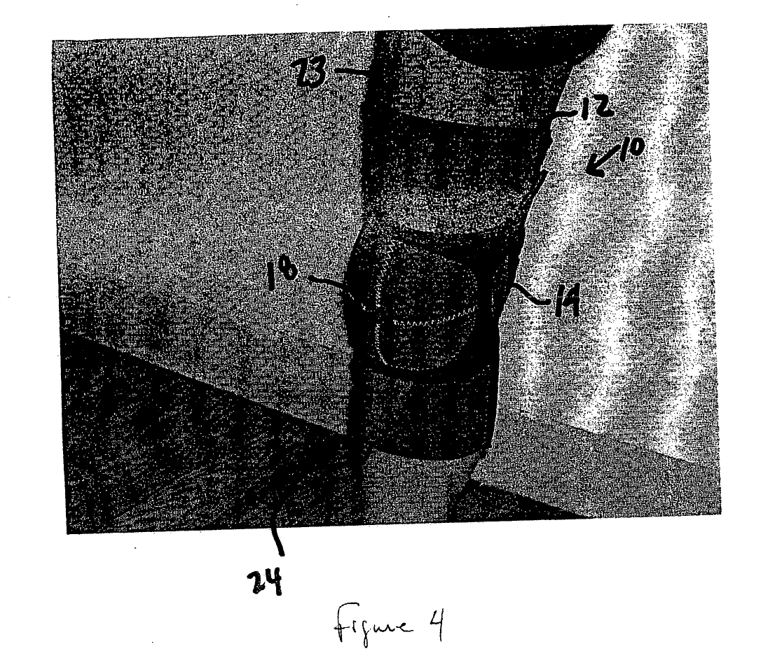

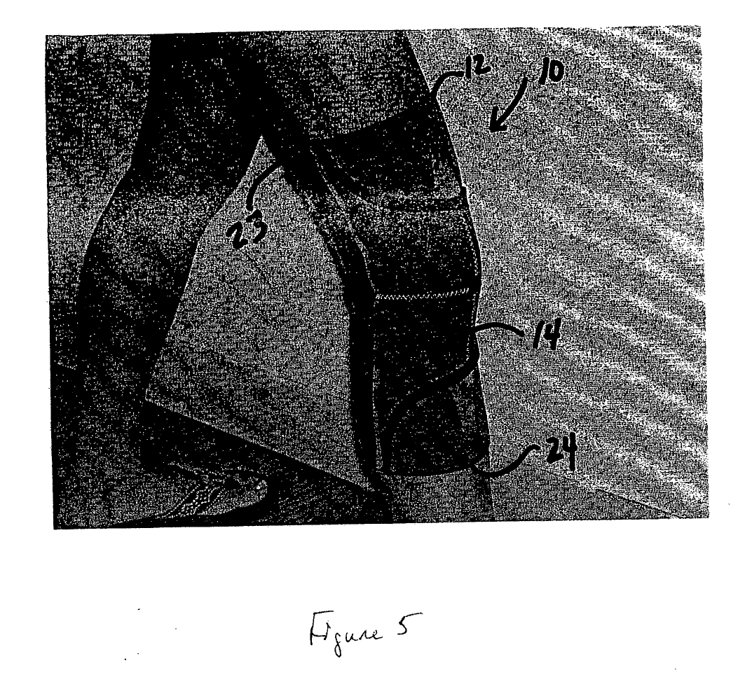

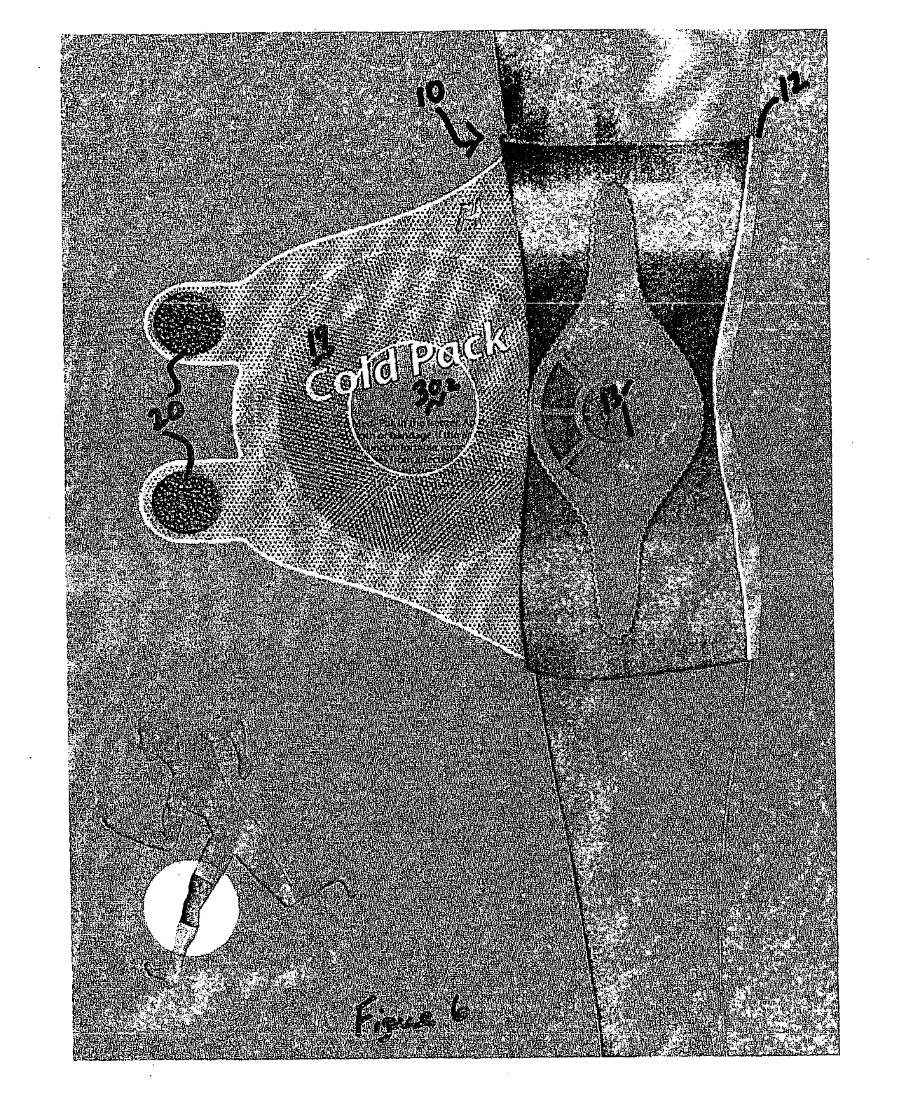

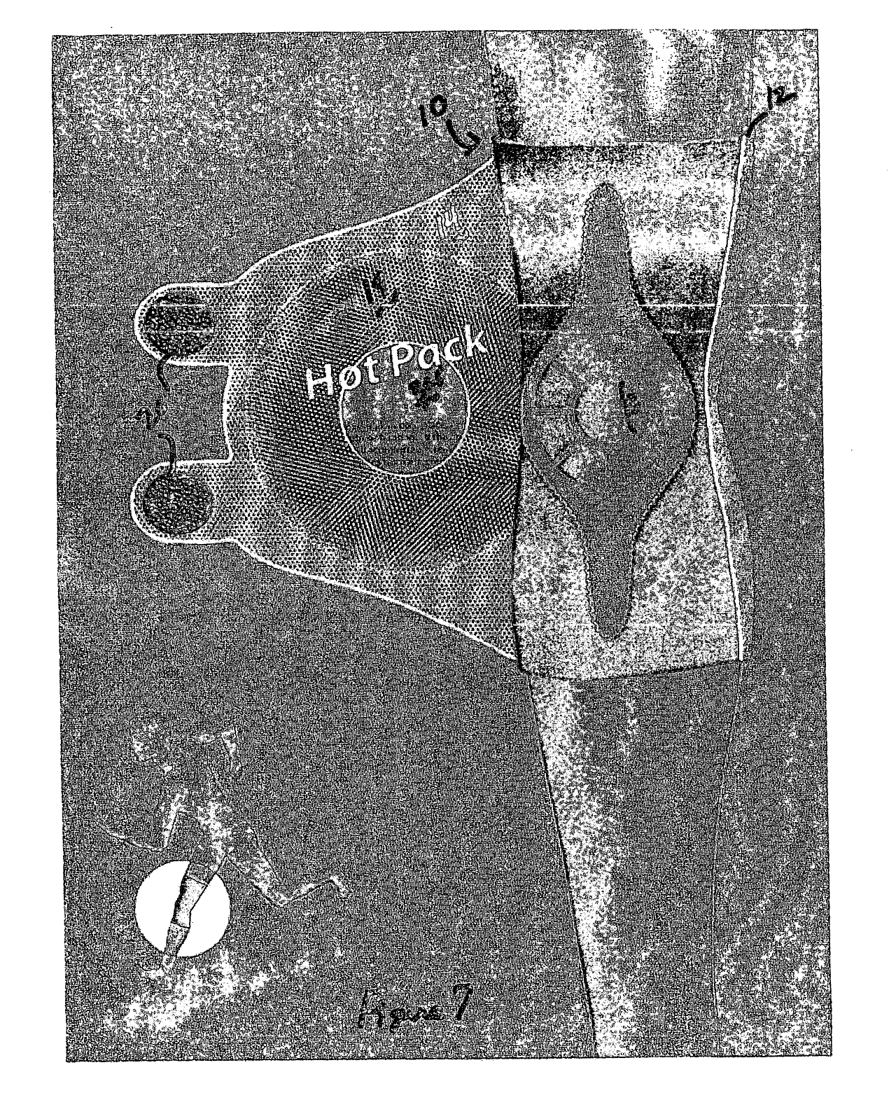

Once a treatment device is either secured to web 13, or inserted in cup 18 and held in place by liner 19, or alternatively merely positioned in place, the overwrap 14 is closed over the web 13 and about the sleeve 12 as shown in FIG. 2. FIG. 3 shows a photograph of a knee support similar to that illustrated in FIG. 1, while FIG. 4 shows the knee support 10 in its closed position similar to the illustration of FIG. 2. FIG. 5 shows the knee support in use after the overwrap has been secured. FIGS. 6 and 7 illustrate the positioning of a therapeutic delivery device such as cold pack 30a in FIG. 6 or hot pack 30b in FIG. 7 within the cup 18 and liner 19. Alternatively, in the absence of a liner 19, cold pack 30a or hot pack 30b may simply be placed over web 13. When overwrap 14 is wrapped about the sleeve 12 of knee support 10, these therapeutic packs 30a, 30b are held in position over the web 13 and temperature therapy is applied to the region of the wearer's knee.

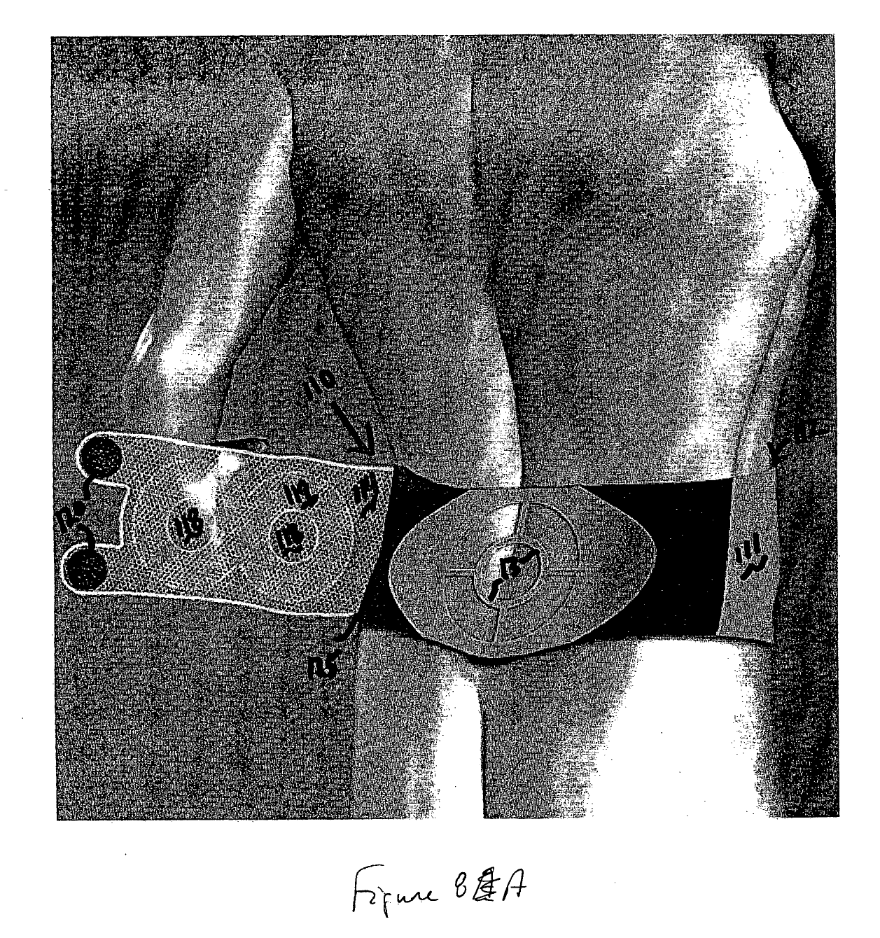

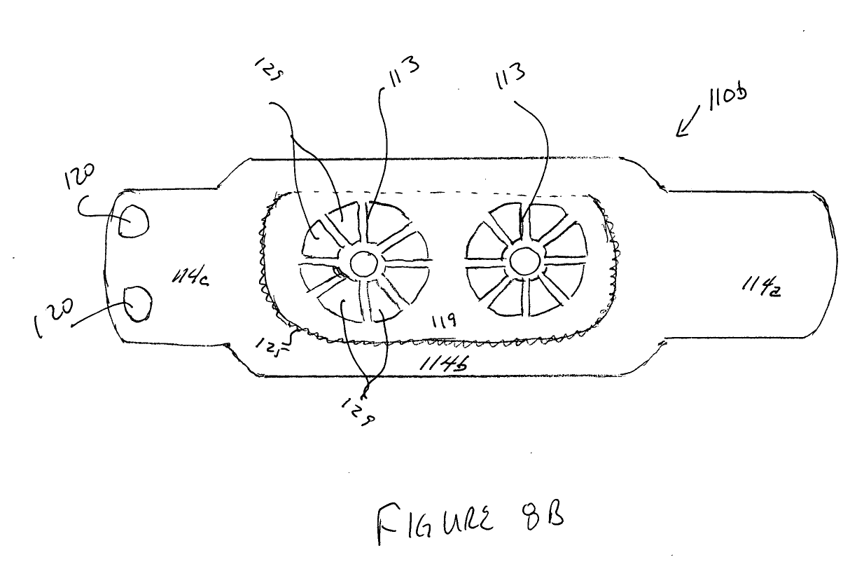

FIG. 8a depicts a similar support 110 specially designed for use on the wearer's torso, and particularly to administer therapy to the wearer's back. Support 110 comprises a brace body 111 formed into a sleeve 112 which is preferably elasticized and slipped onto the wearer's body. However, as shown in FIG. 8b, particularly in the case of back support 110b, the brace body may not be secured into a fixed sleeve, but may be left in sheet form for attachment by the wearer using hook and loop fasteners 120 or the like. The wrap form back support 110b comprises a wrap 114 with a first edge 114a, a body 114b, and a trailing edge 114c with fasteners 120. A liner 119 is attached to body 114b as by seam 125 along the sides and bottom. No web structure is required to maintain strength because wrap 114 has no external openings. Instead, web patterns 113 are placed in liner 119. In this fashion, liner 119 forms a pocket to hold therapeutic devices 30, while providing openings to permit the therapeutic devices to contact the wearer's skin. The wrap-style brace of FIG. 9 illustrates another structure for positioning a therapeutic device 30 next to web 113.

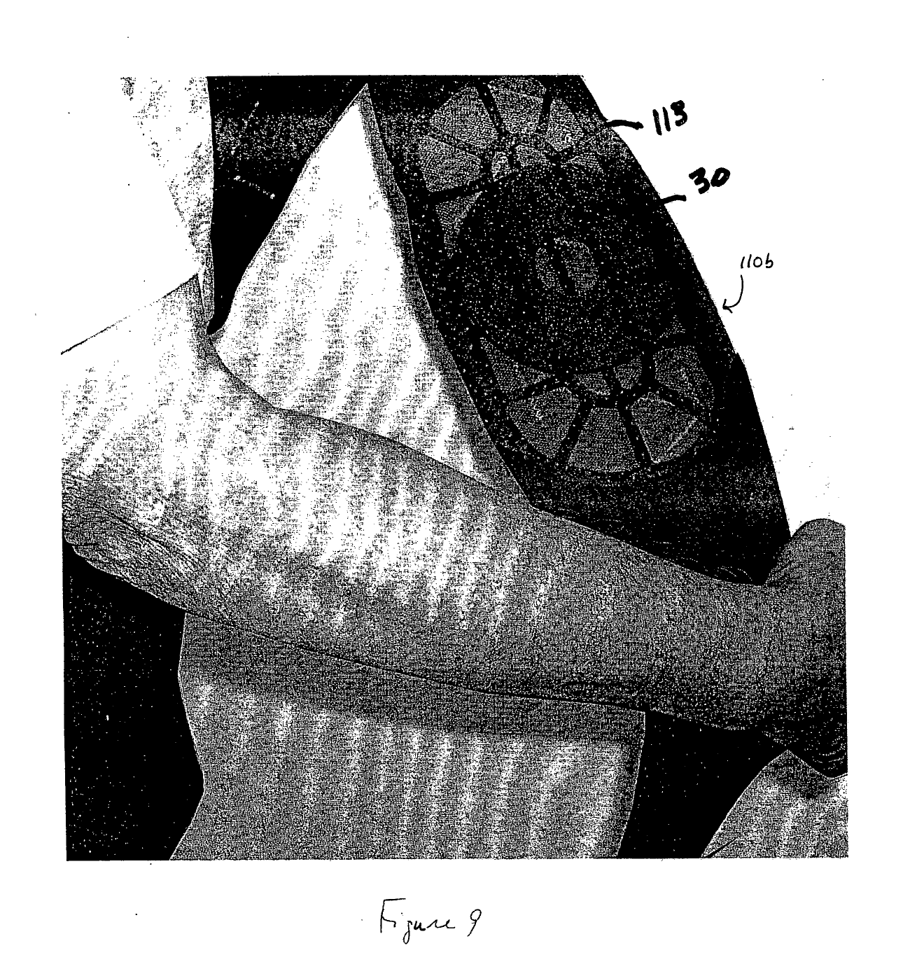

FIG. 10 depicts yet another support according to the present invention, in this case an ankle brace 210 is formed from brace body 211 and creates a sleeve 212. In this instance, sleeve 212 is not purely cylindrical but substantially L-shaped with opening 201 to receive the wearer's heel and thereby position sleeve 212 securely at the ankle. Web 213 is again positioned at a location where therapy is to be applied to the wearer, and it is to be understood that the shape of the web 213 may be varied according to the type of therapeutic delivery devices intended to be utilized with the brace 210. Overwrap 214 is attached to sleeve 212 and has cup 218 with liner 219 that may receive therapeutic delivery devices. After therapeutic delivery devices are either attached to web 213, placed on web 213, or placed in cup 218, the overwrap 214 is closed about the ankle and securing means such as hooks 220 attach the overwrap to the sleeve. The liner 219 is not required, but when present forms a pocket into which therapeutic devices 30 may be held until overwrap 214 is fastened. As is the case of the other illustrated support devices, the overwrap 214 not only assists in securely positioning the therapeutic delivery device utilized with the support, but due to the inherent weakness caused by openings in the web 213 portion of the sleeve, the overwrap also preferably both serves as a structural component of the support and provides a method to adjust the compression provided by the support.

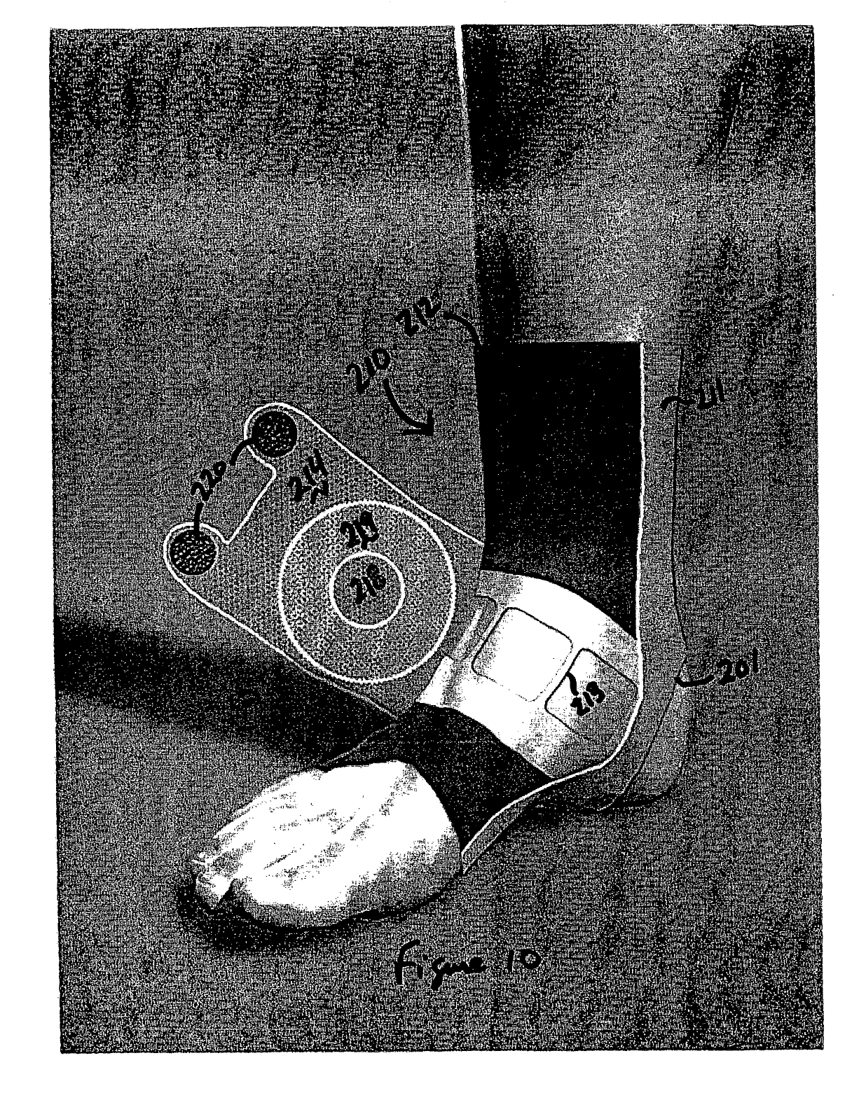

FIG. 11 shows an alternative brace embodiment 310 adapted for use on the arm and elbow. Again, brace 310 is formed from a body 311 fastened into a sleeve 312 forming a lumen which is received on the wearer's arm. Web section 313 is positioned where treatment is to be applied and the treatment device is secured by either web 313 or within cup 318 in the overwrap 314. Once the treatment device is positioned, overwrap 314 is closed about the sleeve 312 and attached to the surface of the sleeve by securing devices such as microhooks 320. If liner 319 is included, it may form a pocket to receive therapeutic inserts that do not engage with web 313.

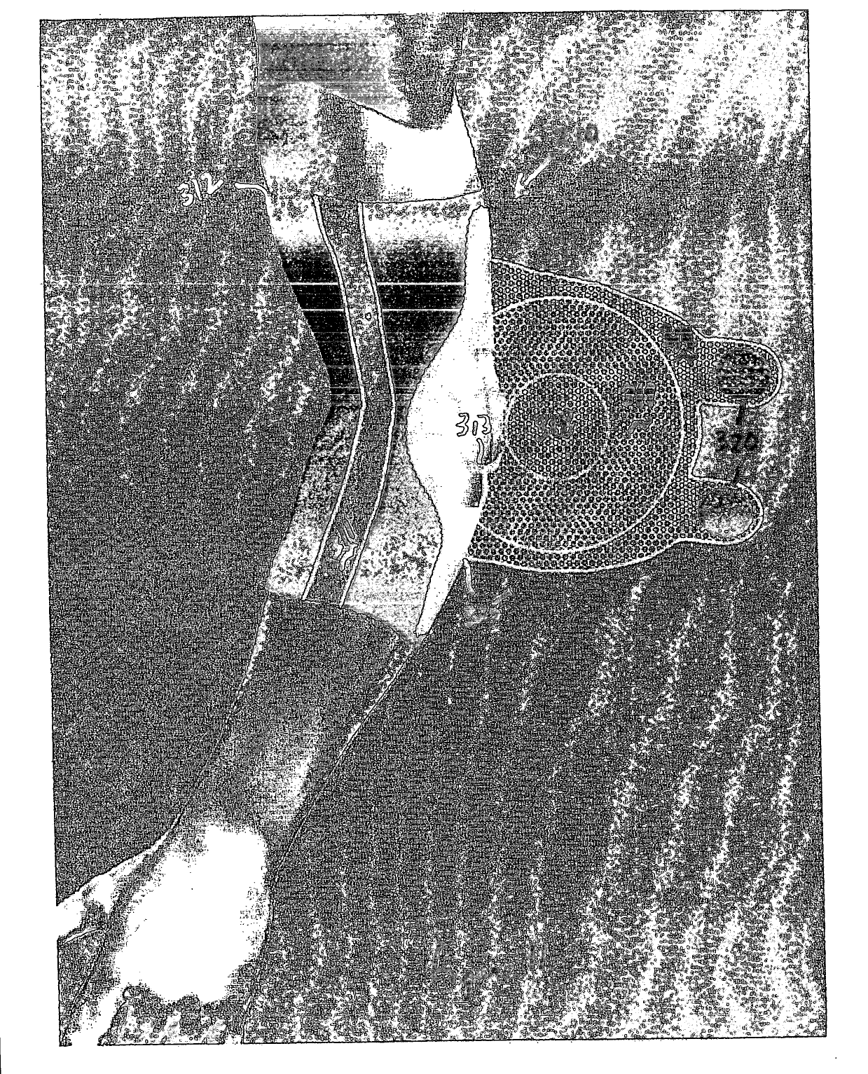

FIG. 12 depicts a wrist support 410 according to the present invention, again formed from a body 411 configured into sleeve 412 to form a lumen and received on the wearer's arm with a web section 413 allowing apertures 429 opening directly to the wearer's skin. Again, a treatment delivery device may be secured either to web 413 or within cup 418 and covered by liner 419 in the overwrap 414. Alternatively, the treatment delivery device may be placed on web 413 so that contact is made with the wearer's skin through apertures 429. When the treatment delivery device is positioned, overwrap 414 is closed about the web 413 and held in place by securing means such as microhooks 420.

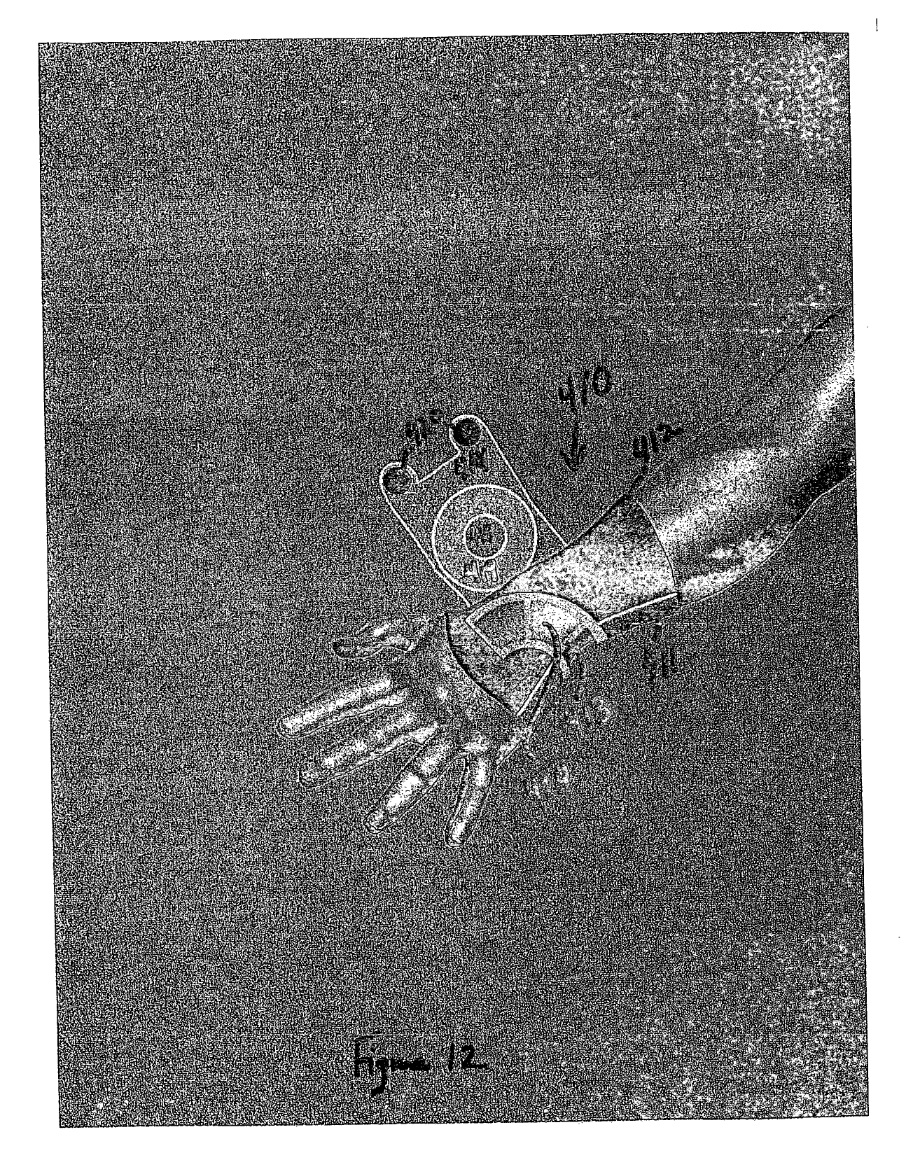

FIGS. 13 and 14 depict a slightly modified embodiment of wrist brace 410 having an opening 402 in the sleeve 412 to receive the wearer's thumb and thereby more securely position sleeve 412 in place at the wrist. FIG. 13 depicts the wrist support 412 in its open treatment delivery device receiving form, and FIG. 14 depicts wrist support 410 in its closed position.

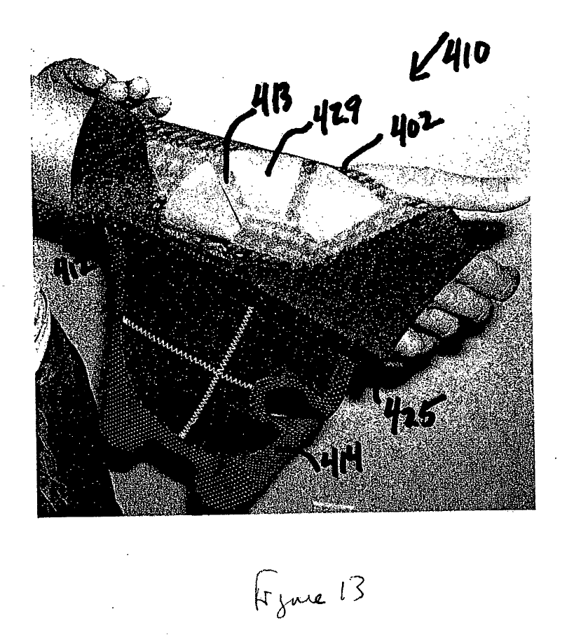

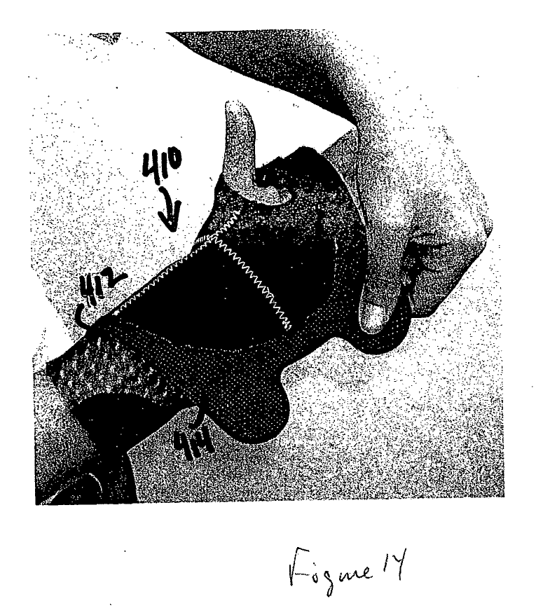

FIGS. 15 through 18 show structures of a family of webs that may be utilized in supports according to the present invention. FIGS. 15a and 15b depict web 13 designed for use in a knee support 10 such as that shown in FIG. 1. The web 13 comprises an inner ring 34 and outer ring 35 connected by ribs 33 and defining uniformly shaped apertures 29 therebetween. At opposite sides of the outer ring 35 the web section a border extends to ends 36 and may be joined with or formed as a portion of the brace body 11 (not shown).

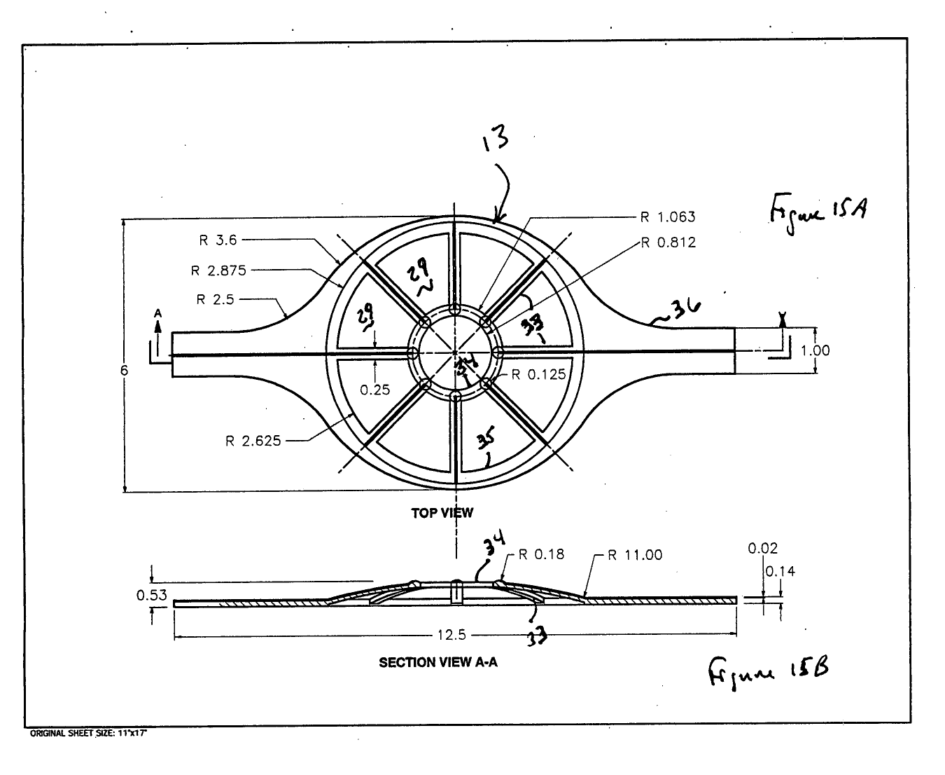

Similarly, FIG. 16 is a top plan view of a web configuration suitable for use in the in elbow support device 310 according to the present invention. The web 313 is comprised of an inner arcuate section 334 and outer arcuate section 335 connected by ribs 333 and defining uniformly shaped apertures 329 therebetween.

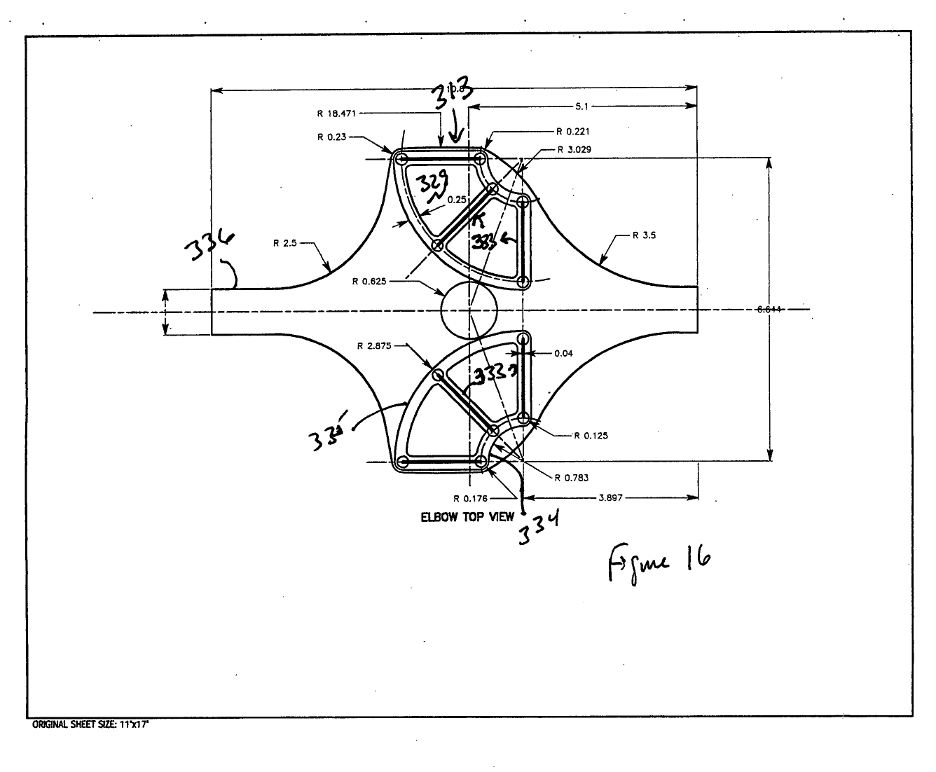

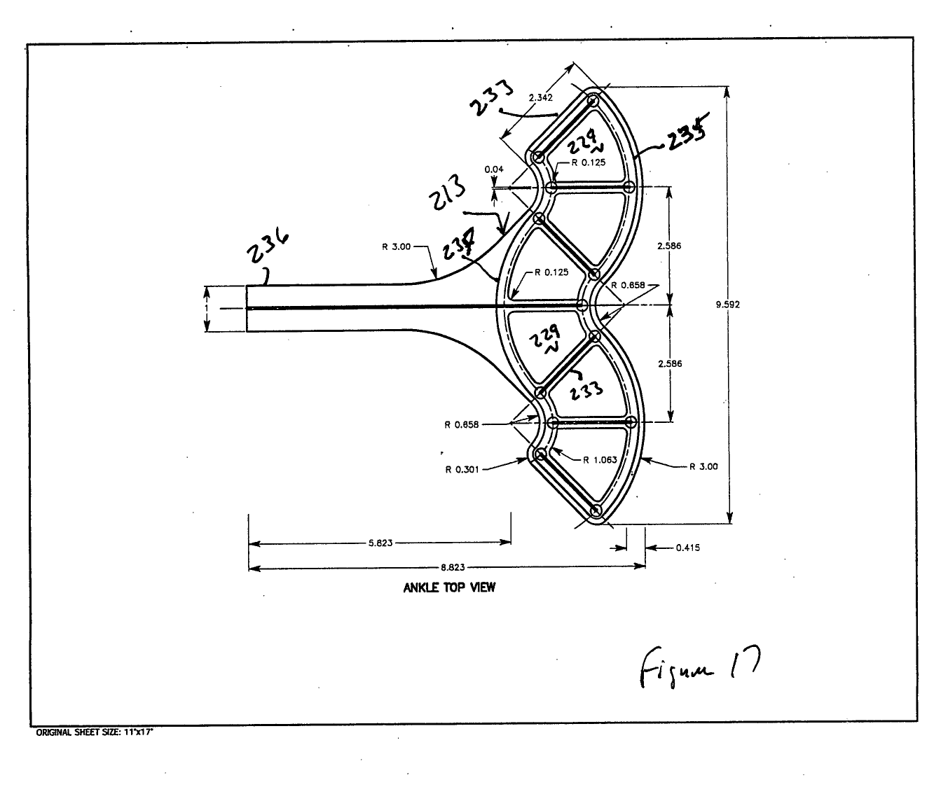

In FIG. 17, a web 213 is shown optimized for use with an ankle support 210 according to the present invention such as that shown in FIG. 10. The web 213 comprises an inner curved section 234, an outer curved section 235 which are almost sinusoidal in appearance but connected by ribs 233. The ribs 233 define openings 229 which are substantially uniform in configuration. Similarly, FIG. 18 shows web 413 optimized for use in connection with wrist support 410 according to the present invention such as that depicted in FIG. 12. Again, inner curved segment 434 and outer curved segment 435 are connected by ribs 433 and defining uniformly shaped apertures 429.

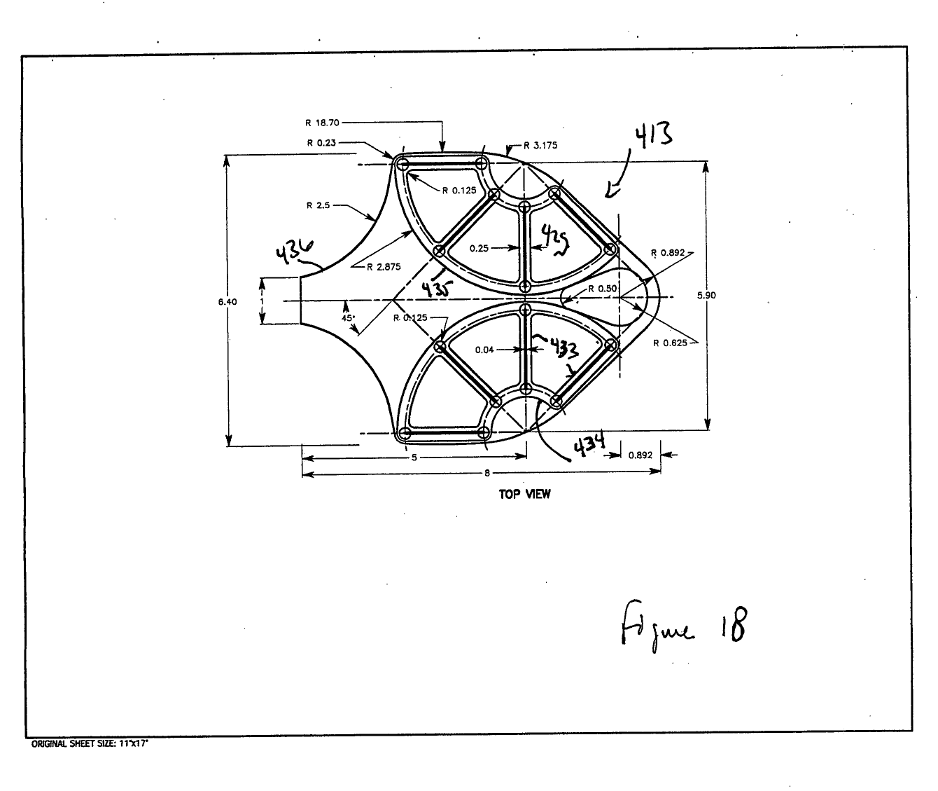

FIG. 19 illustrates two constructions of web 13 and three methods of joining therapeutic medicament dispensing packs 40 to the web 13. The web 13 may advantageously be made of a laminate with a nappy lycra surface 15 that microhooks may attach to, over a conformable foam/wax core 16 with a lycra bottom surface 17. Alternatively, the web 13 is shown constructed of a non-tearable foam with a smooth surface. This foam is stitchable and may be supplied in a variety of colors, as may be lycra 15, 17, or, the foam may even be translucent.

To join the medicament dispensing packs 40 to the web 13, the medicament dispensing packs 40 are constructed of a series of medicament dispensing sections 41 joined by a backing 42. The backing 42 is typically a nonwoven to prevent medicaments, such as typical analgesics, from migrating into the overwrap 14. Intermediate the medicament dispensing sections 41 which might be a permeable foam impregnated with medicament, are intermediate sections 45 which may or may not be perforated 43 to permit separation of the medicament dispensing sections 41. In the intermediate area 45 between medicament dispensing sections 41, one securing option is to provide microhooks 44a that will attach to nappy Velcro 15 on ribs 33 of web 13. Another securing alternative is to have a cohesive foam surface 44b that forms a bond with cohesive foam surface on non-tearable foam ribs 33. Alternatively, the intermediate section 45 between medicament dispensing portions 41 can be made flexible so as to allow medicament dispensing sections 41 to pinch around the ribs 33. Yet another alternative is for the medicament dispensing device 40 simply to interfit with web 13 almost like puzzle pieces to hold device 40 in place while an overwrap is secured to hold the device 40 during the wearing of the brace.

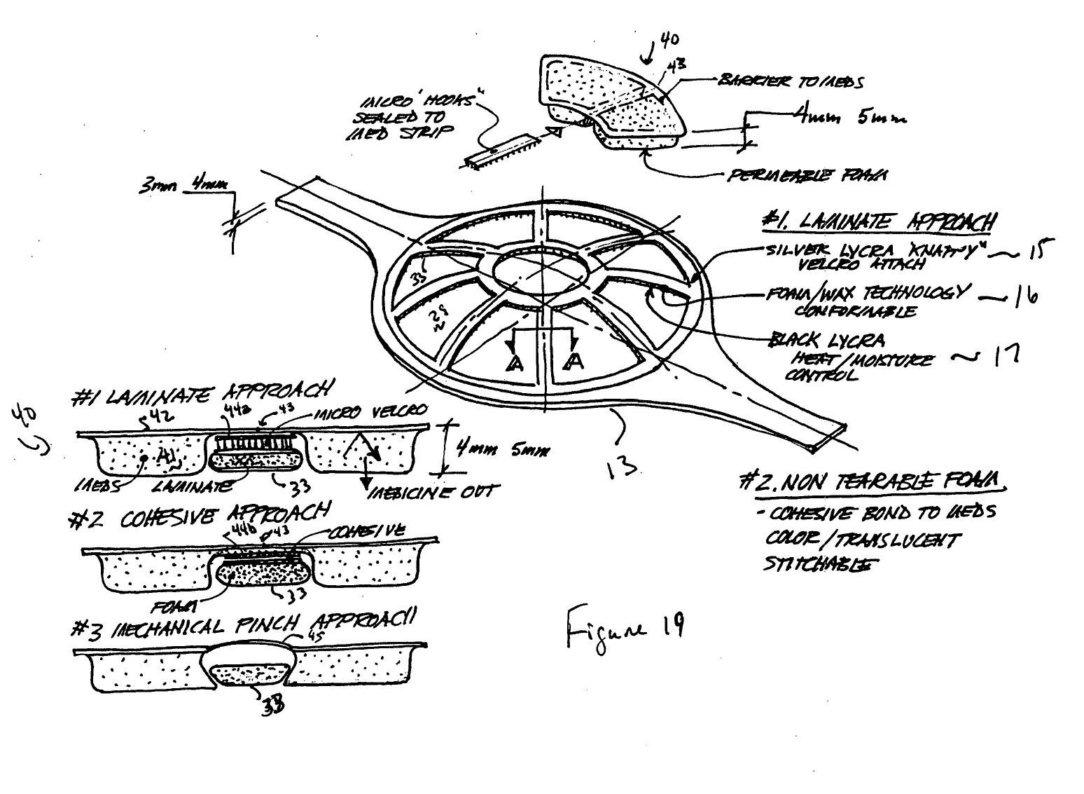

FIG. 20 illustrates one possible laminate structure with top nappy lycra 15, intermediate wax filled foam 16, and bottom lycra 17 used to form the body 11 of knee support 10. The left edge 21 and right edge 22 of body 11 are joined to form a sleeve as depicted in FIG. 1. The overwrap 14 is joined along one edge 25 to the body 11 and has a cup 18 and securing means such as Velcro 20.

FIG. 21 illustrates a mechanical pinch configuration for therapeutic delivery devices such as medicament packs 40 in greater detail with the intermediate section 45 forming a hinge to allow to medicament sections 41 to be bent apart and the hinge section 45 placed on rib 33 of web 13. When the medicament sections 41 are released, they conform slightly around the rib 33 and hold the treatment pack 40 in place on web 13. One disadvantage to mechanical hinge configuration is that medicament packs 40 must always be applied in pairs of medicaments sections 41 and groups of four or eight medicaments sections 41 must be separated into pairs and then applied. Furthermore, single medicaments sections 41 cannot be attached to web 13 with the rib pinching technique.

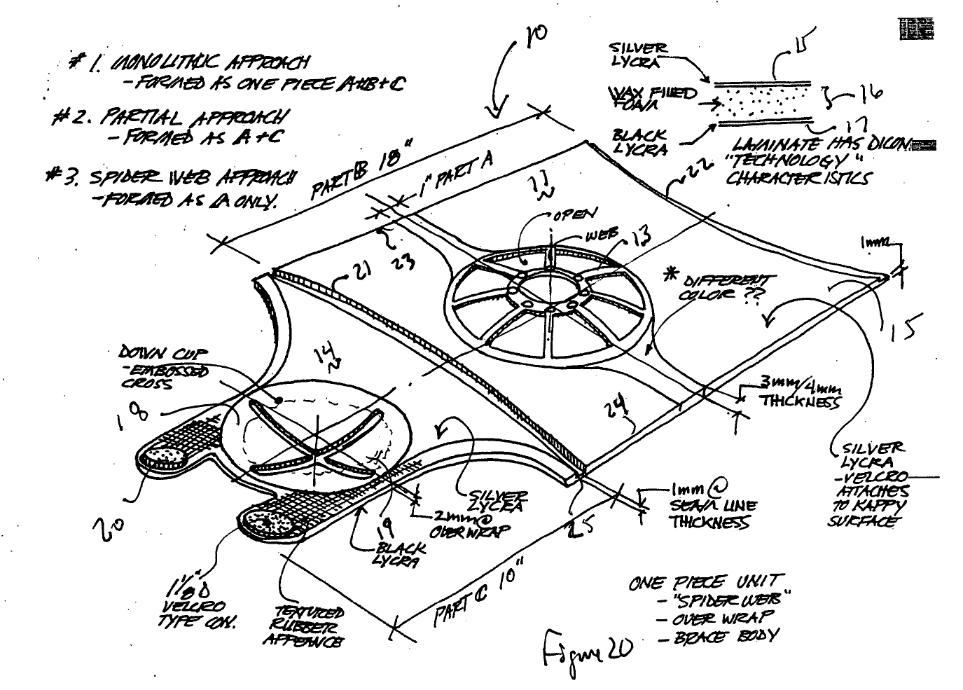

Another alternative for medicament packs 40 is illustrated in FIG. 22 where packs 40 have a medicament dispensing section 41 and backing film 42 where, at least around the periphery of the backing film 42 the film forms a semi-rigid downwardly shaped edge 44. The ribs 33 of web 13 are formed from foam with upward shaped openings 45 that are adapted to receive the downward edges 44 of the medicament packs 40. This structure effectively permits edges 44 the medicament packs 40 to snap on to the ribs 33 defining openings 29 in the web section 13. In each instance, it can be seen that the uniform shape of the medicament packs 40 and medicament dispensing sections 41 permits those sections to be utilized in a variety of web configurations such as those shown in FIGS. 15 through 18. The packs 40 may be supplied in the form of rings of 8 sections 41 but may be separated into groups of two or three sections and attached to or about the ribs 33 of various web configurations.

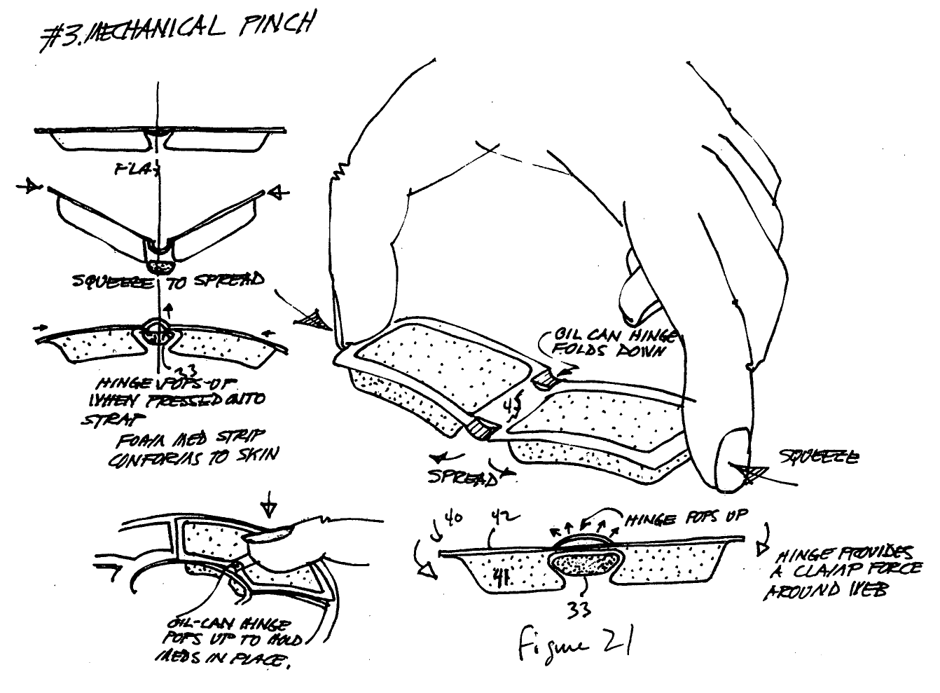

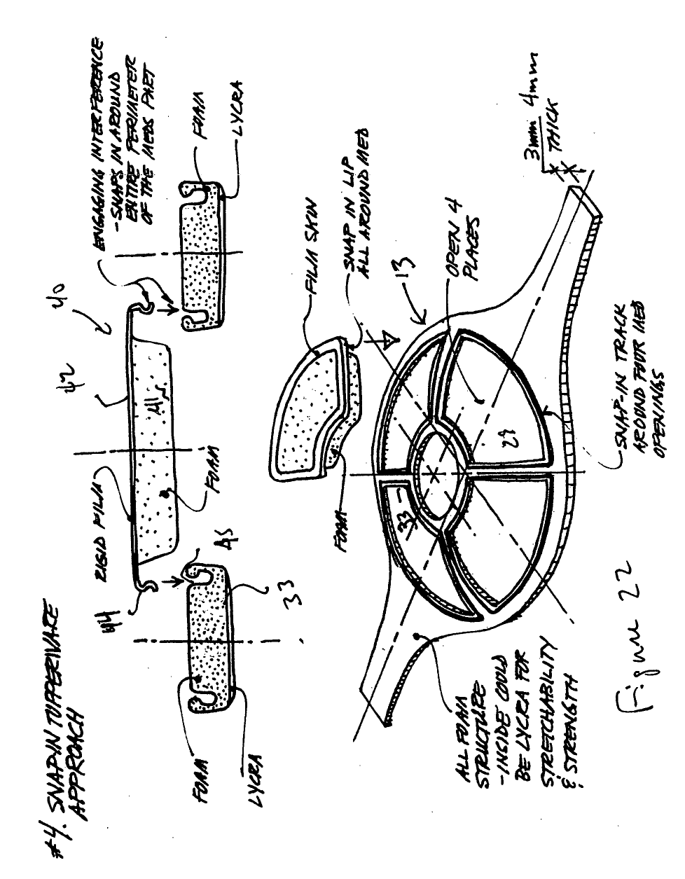

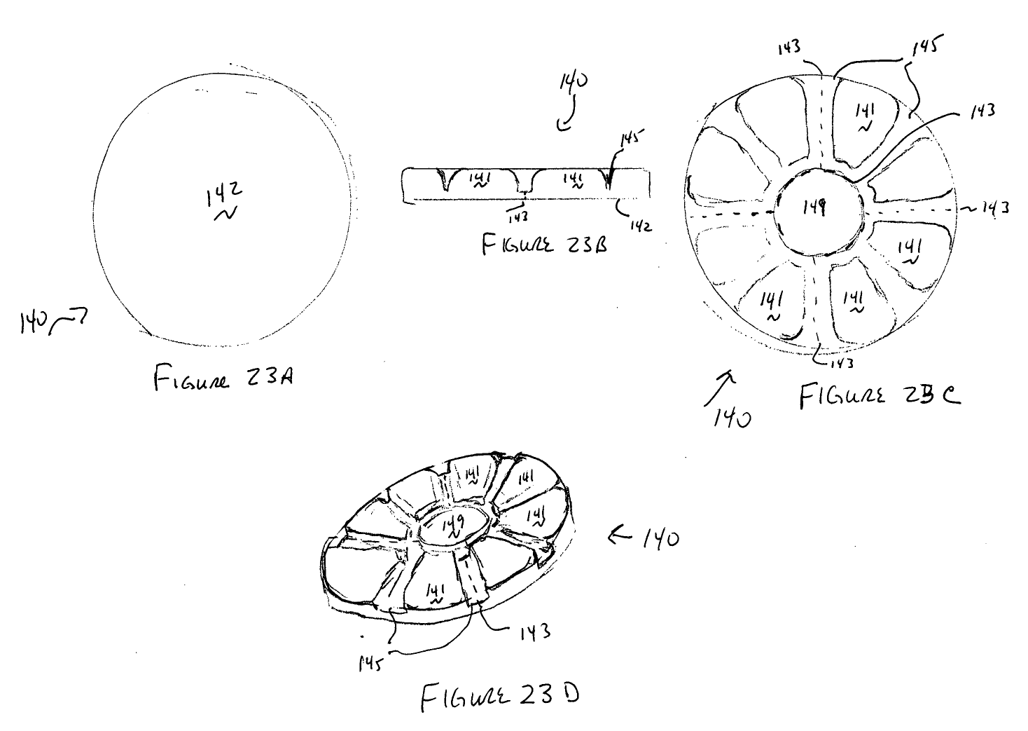

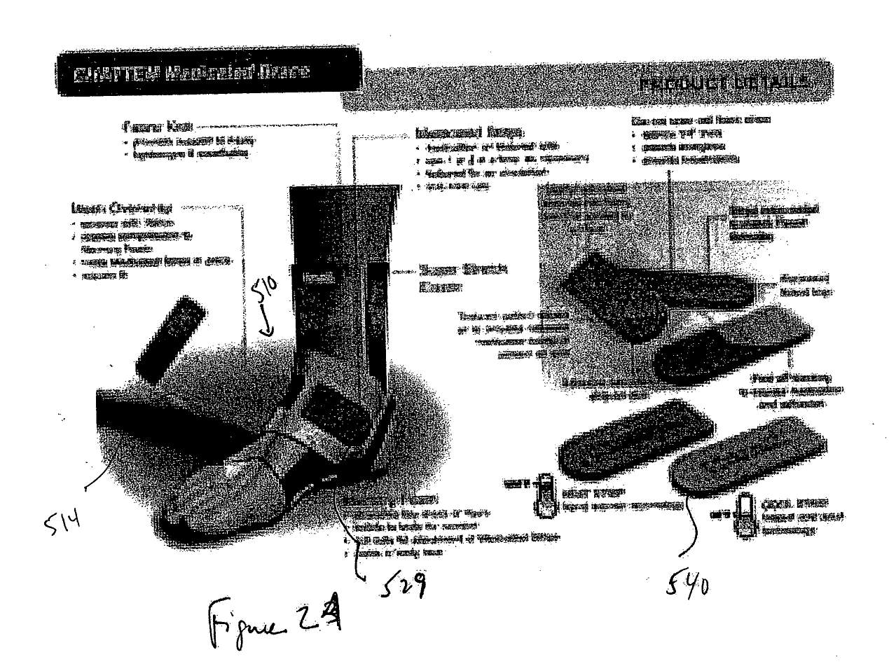

A preferred medicament treatment device 140 is shown in FIGS. 23a-23d. FIG. 23a shows the bottom 142 of the device 140, which may be advantageously formed of hydrophilic foam to capture active or volatile ingredients as described in Palinczar, U.S. Pat. No. 4,339,550 which is incorporated herein by reference. A preferred active and volatile ingredient for pain relief is menthol or its natural precursor, peppermint oil. FIG. 23b shows a side plan view of treatment device 140 where sections 141 are shown divided by channels 145. A top plan view in FIG. 23c reflects the eight somewhat triangular or pie slice shaped sections 141 and central section 149 separated by channels 145. Optimal perforations 143 are shown around central section 149 and between each pair of triangular sections 141. A perspective view is shown in FIG. 23d. The perforations permit the device 140 to be separated to fit in configurations where the web 13 does not form a complete circle. The channels 145 fit upon the ribs 33 of webs 13 and act to constrain the movement of the adjacent sections 141. When further secured by a tensioned overwrap 14, the device 140 or sections 141 are held in position during wearing of the brace. Medicament containing devices 140 are preferably packaged in resealable foil pouches to provide adequate shelf life and to permit unused sections 141 to be resealed and stored for an intermediate length of time for future use. It is preferred that the medicament containing device be generally dividable into fourths or even finer increments to fit various web and aperture configurations. To allow the use of curved aperture configurations as shown in FIGS. 12 and 15-17, the preferred device shape is that of a disk having generally triangular sections 141 separated by radial channels 145.

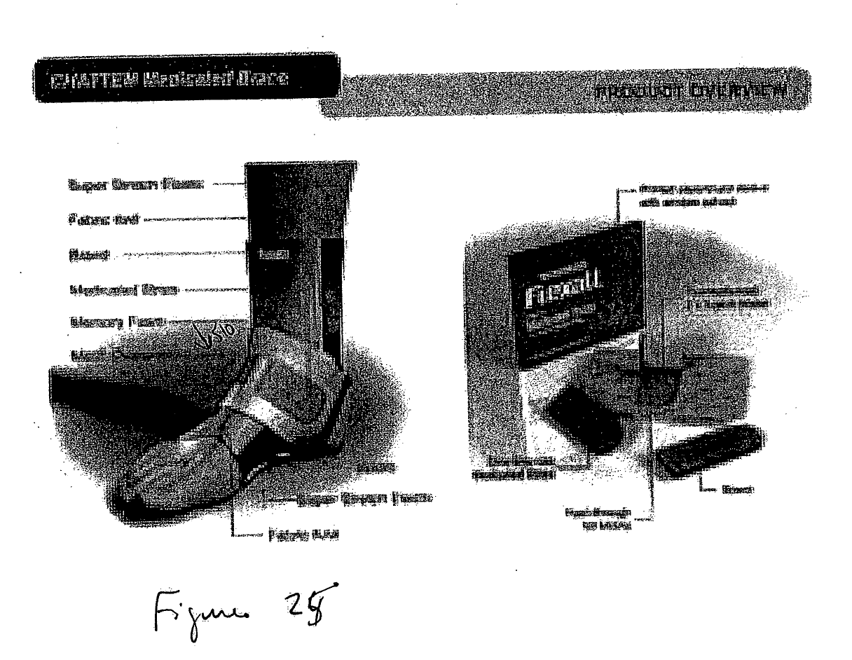

FIGS. 24 and 25 show yet another embodiment of an ankle brace 510 with two openings 529 to receive treatment delivery devices 540. In this case, the treatment devices may adhere directly to the wearer's skin as is common with analgesic patches in the market today. Alternatively, the devices 540 may simply fit into openings 529 and be held in place by overwrap 514.

Although preferred embodiments of the present invention have been disclosed in detail herein, it will be understood that various substitutions and modifications may be made to the disclosed embodiment described herein without departing from the scope and spirit of the present invention as recited in the appended claims.

1. A support device for holding a therapeutic delivery device comprising a sleeve having a wall defining a lumen to receive a wearer's extremity;
a web in the wall having a plurality of apertures separated by a rib;
a therapeutic delivery device positioned over the web;
an overwrap having a front end fastened to the wall on a first side of the web, a body extending over the web or therapeutic delivery device and
a second end releasably secured about the sleeve.

2. The support device of claim 1 wherein the therapeutic delivery device has a channel that receives the rib to position the therapeutic delivery device.

3. The support device of claim 2 wherein the therapeutic delivery device contacts the wearer's skin through the apertures.

4. The support device of claim 1 wherein the overwrap has an interior liner that forms a pocket.

5. The support device of claim 4 wherein a temperature therapy device is placed in the pocket.

6. The support device of claim 3 wherein the therapeutic delivery device is a polyurethane foam containing a medicament.

7. The support device of claim 6 wherein the medicament is menthol.

8. A medicament treatment device for use with a support device having a web with a rib defining a plurality of apertures, said treatment device comprising a plurality of sections divided on a front side by a channel and joined at the back side wherein the channel is adapted to be received on the rib and thereby position the sections within the apertures.

9. The medicament treatment device of claim 8 wherein the device comprises a disk having eight radial channels defining eight sections.

10. The medicament treatment device of claim 8 comprising a hydrophilic polyurethane foam carrying an active ingredient.

11. The medicament treatment device of claim 10 wherein the active ingredient is menthol.

12. The medicament treatment device of claim 8 wherein the channel is perforated to facilitate the separation of the sections.

13. The medicament treatment device of claim 10 wherein the device is packaged in a resealable foil pouch.

14. A combination support and therapy device comprising a web adapted to contact a human wearer's skin, a therapeutic delivery device positioned over the web, and an overwrap having a first end, a body, and a second end wherein the first end of the overwrap is positioned on a first side of the web, the body of the overwrap holds the therapeutic delivery device against the web, and wherein the second end of the overwrap is releasably fastenable to hold the overwrap in place.

15. The combination support and therapy device of claim 14 wherein the therapy delivery device is a temperature therapy device.

16. The combination support and therapy device of claim 14 wherein the web has a rib defining a plurality of apertures and the therapeutic delivery device has a plurality of sections divided by a channel adapted to be received on the rib and the sections thereby positioned to fit within apertures.

17. The combination support and therapy device of claim 16 wherein the therapeutic delivery device is a disk having eight radial channels.

18. The combination support and therapy device of claim 14 wherein the therapeutic delivery device comprises a hydrophilic polyurethane foam carrying an active ingredient.

19. The combination support and therapy device of claim 18 wherein the active ingredient is menthol.

20. The sleeve of claim 1 wherein the web comprises eight radially extending ribs separating eight generally triangular openings.
